(19) United States
(12) Patent Application Publication
(10) Pub. No.: US 2005/0048118 A1
(43) Pub. Date: Mar. 3, 2005

(54) Modified release venlafaxine hydrochloride tablets

(76) Inventors: Joan Cucala Escoi, Barcelona (ES); Montserrat Gallego Luengo, Barcelona (ES); Inocencia Margallo Lana, Barcelona (ES)

Correspondence Address: MARK R. BUSCHER, P.O. BOX 161, CATHARPIN, VA, 20143, US

(21) Appl. No.: 10/895,997

(22) Filed: Jul. 22, 2004

Related U.S. Application Data
(60) Provisional application No. 60/489,923, filed on Jul. 25, 2003.

(51) Int. Cl. A61K 9/20; A61K 9/24

(52) U.S. Cl. 424/472

(57) ABSTRACT

A modified release tablet of venlafaxine hydrochloride is formed by a core containing a lipophilic matrix and venlafaxine hydrochloride and a water-insoluble, permeable coating thereover.

This application claims the benefit of priority under 35 U.S.C. § 119(e) from U.S. provisional application Ser. No. 60/489,923, filed Jul. 25, 2003.

### BACKGROUND OF THE INVENTION

The present invention relates to a pharmaceutical tablet that provides modified release of venlafaxine hydrochloride.

Venlafaxine, or the compound 1-[2-(dimethylamino)-1-(4-methoxyphenyl)ethyl]-cyclohexanol, is an antidepressant that is sold, as the hydrochloride salt, under the brand name EFFEXOR® (Wyeth Ayerst). The compound is described in U.S. Pat. No. 4,535,186 and a further synthesis thereof is described in U.S. Pat. No. 5,043,466. Venlafaxine hydrochloride is available as an immediate release tablet or as an extended release capsule. Modified release dosage forms, such as extended release formulations, can deliver the drug within a safe and effective range over a longer period of time than a normal immediate release dosage form, thereby allowing for less frequent dosing. The ability to take a drug once a day instead of two or three times a day is a benefit to the patient in terms of convenience and also in terms of compliance. Indeed, a controlled release of the drug can in some cases provide superior efficacy and/or reduced side effects as the potential overdosing/under-dosing (i.e. blood plasma concentration peaks and troughs) of a multi-dose regimen are avoided.

A variety of techniques have been generally developed for modifying release rate. Some of these techniques involve forming a matrix to control and/or slow the release of the active agent through diffusion and/or erosion release of the drug. Others rely on coating layers to control the release, especially in pellets intended for use in capsules.

U.S. patent U.S. Pat. No. 6,274,171 and EP 0 797 991 relate to an extended release dosage form of venlafaxine hydrochloride. Specifically, an encapsulated dosage form is taught that comprises spheroids of venlafaxine hydrochloride, microcrystalline cellulose, and hydroxypropylmethylcellulose (HPMC). These spheroids are coated with a mixture of ethyl cellulose and HPMC. By providing an appropriate amount of the coating, the desired blood plasma profile can be obtained. An acceptable batch of coated spheroids meets the following in vitro dissolution profile:

| | Average % venlafaxine |
|---|---|
| Time (hours) | hydrochloride released |
| 2 | <30 |
| 4 | 30-55 |
| 8 | 55-80 |
| 12 | 65-90 |
| 24 | >80 |

While the encapsulated dosage form disclosed in these patents appears to be suitable for making an extended release dosage form, the dosage form is somewhat complicated and must be an encapsulated form. A tablet would be desirable. However these patents disclose that “[n]umerous attempts to produce extended release tablets by hydrogel technology proved to be fruitless because the compressed tablets were either physically unstable (poor compressibility or capping problems) or dissolved too rapidly in dissolution studies.” See U.S. Pat. No. 6,274,171 at column 4, lines 60-65 and EP 0 797 991A1 at page 3 lines 35-37.

Thus, it would be desirable to provide a tablet that can provide modified release of venlafaxine hydrochloride, especially an extended release venlafaxine hydrochloride tablet that is bioequivalent to the commercial venlafaxine hydrochloride extended release capsules.

### SUMMARY OF THE INVENTION

The present invention relates to the discovery of a modified release tablet that can provide modified release of venlafaxine hydrochloride. Accordingly, a first aspect of the invention relates to a pharmaceutical tablet composition comprising (i) a core, which comprises a lipophilic matrix and an effective amount of venlafaxine hydrochloride; and (ii) a water insoluble, permeable coating over said core. The lipophilic matrix is preferably a fatty acid wax and the water insoluble, permeable coating is preferably comprised of an acrylic polymer such as a methacrylate copolymer.

Another aspect of the invention relates to a process for making a modified release tablet, which comprises (i) granulating venlafaxine hydrochloride and a fatty acid wax to form granules; (ii) compressing the granules optionally with additional excipients to form a tablet; and (iii) forming a water insoluble, permeable coating around the tablet.

A further aspect of the invention relates to a process for a modified release tablet, which comprises: (a) co-processing a fatty acid wax with calcium phosphate to form a modified calcium phosphate; (b) blending the modified calcium phosphate, a venlafaxine hydrochloride, and optionally one or more additional excipients to form a compression blend; (c) compressing the compression blend to form a tablet; and (d) coating the tablet with a water-insoluble, permeable coating.

### DETAILED DESCRIPTION OF THE INVENTION

The modified release tablets of the present invention provide a non-immediate release profile of a pharmaceutical active agent. The modified release thus includes delayed release, extended release, controlled release and/or combinations thereof. As used herein, an immediate release tablet is one that releases 80% of the active within thirty minutes in a dissolution test using 0.1 N HCl. A non-immediate release tablet requires more than thirty minutes to release 80%. In some embodiments less than 80% of the active is released during the first two hours, more typically less than 50%, and preferably less than 30% of the active is released during the first two hours of the dissolution test. In general the modified release tablets of the present invention have a reduced or controlled burst effect. That is, an initial high release rate of the active agent is avoided or reduced. Preferably the modified release tablets are extended release tablets having a relatively constant release rate of the drug; i.e., a zero order release rate or near zero order release rate. In some embodiments, the modified release tablet enables once daily dosing.

The tablet core comprises at least a lipophilic matrix and venlafaxine hydrochloride. The lipophilic matrix is solid, water-insoluble, and non-swellable and serves to hold/delay the active agent from leaving the tablet. The lipophilic matrix-forming agent is preferably a lipophilic fusible compound, more preferably a fatty acid wax. “Fatty acid wax” as used herein means a material, which is solid at room temperature (i.e. 25° C.), that is made of one or more fatty acids and/or esters of a fatty acid(s) with a mono- and/or polyfunctional alcohol, wherein the preferred alcohol is glycerol. Generally the fatty acids have 12 to 30 carbon atoms, more typically 14 to 24 carbon atoms and the esters generally contain 13 to 80 carbon atoms more typically 18 to 60 carbon atoms. Examples of the fatty acid waxes are palmitic, behenic or stearic acid, glyceryl behenate, glyceryl palmitostearate, hydrogenated castor oil or other natural waxes, including microcrystalline waxes. Accordingly, mixtures of various kinds of fatty acids and fatty acid esters as well as mixtures of waxes all fall under the meaning of “fatty acid wax” in the invention. The fatty acid wax normally has a melting point or range that begins within the range of 40° C. to 140° C., more typically 50° C. to 100° C., however such is not required.

The matrix can contain other excipients dispersed therein, but such is not required. The matrix, containing the pharmaceutical active agent, can constitute the entire core of the tablet or the matrix can be mixed with other excipients in forming the core. Typical excipients in the core include diluents, fillers, binders, lubricants, disintegrants, glidants, colorants, pigments, taste masking agents, sweeteners, plasticizers, and any acceptable auxiliary substances such as absorption enhancers, penetration enhancers, surfactants, co-surfactants, and specialized oils. Examples of excipients include calcium phosphates, such as dibasic calcium phosphate, anhydrous dibasic calcium phosphate, tribasic calcium phosphate, etc.; microcrystalline cellulose; lactose; magnesium stearate; and/or talc.

Preferably the core is lipophilic or hydrophobic. Water-soluble and water-swellable excipients are preferably omitted therefrom.

The core contains an effective amount of venlafaxine hydrochloride. Generally the amount ranges from 30 mg to 300 mg in the tablet core. Contemplated doses include amounts of about 37.5 mg, 75 mg, 100 mg, 112.5 mg, 150 mg, 200 mg, and 300 mg strengths. For clarity, the weight of venlafaxine hydrochloride is expressed herein in terms of the weight of the free base contained in the venlafaxine salt compound, as is conventional in the art. In terms of percentage of the core weight, the venlafaxine hydrochloride typically comprises from 5% to 60%, more usually 10% to 50% of the core weight. The lipophilic matrix generally comprises 20% to 80%, preferably 25% to 60% of the core by weight. In some embodiments, the ratio of the venlafaxine hydrochloride to lipophilic matrix constituent(s) is within the range of 0.75-1.3:1, and in some embodiments is about 1:1+/−5%. Usually the other excipients comprise from 0% to 40%, more typically 1% to 20%, the weight of the core.

The coating over the core is a water insoluble, permeable coating. Generally the coating is hydrophilic and water swellable. The coating is permeable in that water can penetrate the coating and the active agent can be released there through. The coating is preferably comprised of an acrylic polymer. Within the invention, an “acrylic polymer” means a pharmaceutically acceptable copolymer of methacrylic acid or a salt thereof and an acrylic or methacrylic acid, acid ester, and/or salt thereof such as sold under brand name Eudragit®. Examples of acrylic polymers include acrylate-methacrylate copolymers, ammonioalkyl methacrylate copolymers and metacrylic ester copolymers. In particular, Eudragit RL, RS and NE series are preferred. The coating can contain one or more acrylic polymers and/or other ingredients including other polymers, surfactants, plasticizers, etc. Normally, the coating consists primarily of one or more acrylic polymers. Preferably the acrylic polymer is one or more selected from the Eudragit RL series (high permeable), Eudragit RS series (low permeable), mixtures thereof, or Eudragit NE series. The amount of coating is usually within 3% to 45%, more typically, 8% to 35%, the weight of the core. The appropriate amount will depend on the core, the active agent, the kind of materials in the coating and the desired modification of the release.

The coating need not be in physical, direct contact with the core as intervening layers may instead be present, so long as the coating surrounds or envelops the core. Preferably the water insoluble coating is disposed on the core.

In general, the water insoluble, permeable coating is especially useful for controlling the venlafaxine hydrochloride release during the initial dissolution/ingestion time frame. Lipophilic matrices can sometimes suffer from an initial burst effect as active agent located on and/or near the core surface is not sufficiently delayed in leaving the tablet. The water insoluble, permeable coating used in the present invention generally mitigates this burst effect.

In addition to the water insoluble, permeable coating, the tablet of the invention can further contain an enteric coating over the water insoluble, permeable coating. Such a coating is useful for providing acid protection as is conventional in the art, and dissolves in the higher pH environment of the intestines.

Any tabletting process that provides a lipophilic matrix core and a coating can make the modified release tablets of the invention. A convenient process comprises (i) granulating, preferably melt granulating, a pharmaceutical active agent and a fatty acid wax to form granules; (ii) compressing said granules optionally with additional excipients to form a tablet; and (iii) forming a water insoluble, permeable coating around said tablet.

Melt granulation is well known in the art and generally comprises mixing a fusible carrier in a molten state with the active agent to form a partially melted mass and then cooling the mass upon stirring to form a solidified product. A fusible carrier used herein is a lipophilic matrix forming material containing fatty acid wax.

Typically the venlafaxine hydrochloride, fatty acid wax, and optionally one or more fillers, antiadherent agents, lubricants, etc. are combined or mixed in a granulator. The materials are then heated by any convenient technique, such as by a heating jacket, microwaves, infrared, etc. or a combination of two or more techniques. The mixture is heated to a temperature near or above the melting or softening point of the wax, thereby allowing the fusible carrier to act as a liquid binder. If two or more fusible carriers are present, such as two lipophilic matrix forming materials, the temperature need only reach near or above the melting or softening point for one of the carriers. This state is considered a “molten” state for purposes of the present invention. The venlafaxine hydrochloride does not have to melt during this heating or mixing step. The molten fusible carrier is mixed with the venlafaxine hydrochloride and any additional optionally combined excipients to form a partially melted mass. Preferably the mixing is sufficient, given the degree of melting/fluid state of the carrier, the relative amounts of the carrier, the venlafaxine hydrochloride, and any other excipients present, to form a substantially uniform mixture of the combined ingredients. The mixing is preferably carried out using an impeller and a chopper (stirring blades). While the ingredients are normally combined in a non-molten state and mixed before as well as during the heating step, such is not required. For example, the fusible carrier in molten state can be directly combined and mixed with the venlafaxine hydrochloride. Similarly, the mixing may begin only after the fusible carrier starts to soften, partially melt, or completely liquefy.

Granules are formed by the mixing action optionally with cooling. The cooling can be passive, i.e. by removing the heat source, but more typically involves applying a cooling technique such as cool water through a jacket surrounding the granulation bowl and/or with gas transmission through the bowl mass. The granules thus contain the lipophilic matrix having the venlafaxine hydrochloride dispersed therein.

The granules are then compressed to form a tablet, optionally after being first combined with additional excipients. The additional excipients may comprise fillers, binders, lubricants, colorants, etc. Optionally, a second portion of the matrix-forming agent used in formation of granules may be added as the additional excipient to the pre-compression blend. In this way, the release profile of the tablet may be further modified.

In an alternate process, the venlafaxine hydrochloride and the matrix-forming agent and, optionally, other ingredients are mixed in a solid state and then directly compressed into a tablet form; i.e., a so-called direct compression technique. To make the lipophilic matrix easier to handle, especially a fatty acid wax, it is generally convenient to first co-process the matrix material with a flow enhancer to form a modified excipient. Flow enhancers include silica, calcium phosphate, and calcium gluconate, but are not limited thereto. The co-processing is not particularly limited and generally is any process that achieves an improvement in the flowability/handling of the lipophilic matrix material. For example, the flow enhancer can be melt granulated with the wax material to form matrix granules as described in U.S. Pat. No. 5,169,645. Alternatively, the matrix material can be coated on and/or combined with the enhancer, especially calcium phosphate, to form particulates. Examples of such a modified excipient made from fatty acid wax and calcium phosphate are described in U.S. application Ser. No. 10/882,669, filed Jul. 2, 2004.

The modified excipient is then blended with the venlafaxine hydrochloride optionally with other excipients to form a compression blend. The blending can be done in one or more steps. The compression blend is then compressed into tablets. The compression causes the venlafaxine hydrochloride to be effectively dispersed within the lipophilic matrix material, thereby forming the core of the tablet.

The tablets can be any convenient size or shape and typically are within the range of 4 to 20 mm, more typically 4 to 8 mm based on a round tablet. The shapes include round, oval, octagonal, etc. and can be flat, biconvex, and/or scored, but are not limited thereto.

The coating can be formed by any suitable technique, generally spray, drum, or pan coating as are well known in the art. The coating is normally applied as a polymer latex, i.e. an acrylic polymer latex such as Eudragit RL, RS, NE, etc.

The tablet delivery system of the invention preferably allows for controlled release of venlafaxine hydrochloride. More particularly, the venlafaxine hydrochloride is released from the tablet for at least 12 hours, wherein the initial burst is minimized. It is possible to obtain even a zero-order release, by selection of proper amount of the matrix-forming agent in the core and proper composition and amount of the coating. Without wishing to be bound by theory, it is believed that the delivery system properly acts due to synergism between the hydrophobic/lipophilic matrix core and the water insoluble but permeable hydrophilic film in the coating. This combination can provide for zero order or near zero order release of venlafaxine hydrochloride, even when used in relatively high amounts.

The venlafaxine hydrochloride-containing tablets made according to the present invention preferably meet the following in vitro dissolution profile:

| | Average % venlafaxine |
|---|---|
| Time (hours) | hydrochloride released |
| 2 | <30 |
| 4 | 30-55 |
| 8 | 55-80 |
| 12 | 65-90 |
| 24 | >80 |

using USP Apparatus 1 (basket) at 100 rpm in purified water at 37° C. Preferably, an extended release dosage form meets the above dissolution profile using a two media dissolution test. Specifically, during the first two hours, the media is a simulated gastric fluid (SGF) of pH 1.2 while during the remaining hours the media is a simulated intestinal fluid (SIF) of pH 6.8. This two media test can provide more accurate predictions of in vivo performance in some circumstances, especially when an enteric coating is present on the dosage form. Most advantageously the extended release dosage form meets the above dissolution profile in both 0.1N HCl aqueous solution as well as pure water.

For purposes of the present invention, the simulated fluids are defined as follows: SGF (USP Simulated Gastric Fluid without pepsin) composition:

| | | |
|---|---|---|
| HCl | qs | pH 1.2 |
| NaCl | | 0.2% |
| water | qs | 1000 ml |

SIF (USP Simulated Intestinal Fluid without pancreatin) composition:

| | | |
|---|---|---|
| KH₂PO₄ | | 6.8 g |
| NaOH | qs | pH 6.8 |
| water | qs | 1000 ml |

In this regard, a preferred composition is bioequivalent to the commercially available EFFEXOR XR®.

Each of the patents and applications mentioned above are incorporated herein by reference. The invention will be further described with reference to the following non-limiting example.

### EXAMPLE 1

Venlafaxine Hydrochloride

Core (Uncoated Tablets)

Formulation

| | Weight (mg) |
|---|---|
| Venlafaxine hydrochloride | 42.42 mg |
| Glyceryl behenate (Comprito ® 1 ATO 888) | 42.42 mg |
| Anhydrous dicalcium phosphate (A-tab ®) | 14.16 mg |
| Magnesium stearate | 1.00 mg |
| Silicon dioxide (Aerosil ® 200) | 0.50 mg |
| Total weight | 100.50 mg |

Process

Mixing: Glyceryl behenate and Venlafaxine hydrochloride were mixed together in a high shear mixer Mi-Mi-Pro (Manufactured by Pro-C-epT, Belgium) using impeller at 250 rpm and chopper at 200 rpm for 3 minutes.

Granulation: The oven temperature of the high shear mixer was limited to room temperature (15° C. programmed) and microwaves were used as the unique source of heating (vacuum: 400 mbar, impeller: 400 rpm, chopper: 350 rpm). The product temperature increased up to 69-70° C., reaching a maximum torque of 28%. Then, an intensification phase was carried out (impeller: 800 rpm, chopper: 700 rpm). A granulate was formed.

Sieving: The granules were sieved manually, first through a 1.4 mm screen and after by 710 microns screen.

Compression: Anhydrous dicalcium phosphate and silicon dioxide were added to the granules and this mixture was sieved through 710 microns screen and mixed in a Turbula mixer for 15 minutes. Magnesium stearate was added to the mixture and mixed in a Turbula mixer for 5 minutes. Compression was carried out in an instrumented Korsh tablet eccentric press EK0 obtaining biconvex round 5 mm diameter, 100 mg weight tablets.

Coating

Formulation

| | Coating suspension |
|---|---|
| Eudragit RL 30 D | 1000.2 g |
| Triethyl citrate | 60 g |
| Magnesium stearate | 45 g |
| Simethicone emulsion 30% | 1.5 g |
| Purified water | 100.8 g |
| Total coating suspension | 2107.5 g |

Process

Coating: The tablets were coated in a BLC 5/10 (coating equipment) manufactured by L. B. Bohle, Germany.

The coating parameters were:

| | |
|---|---|
| Drum speed: | 15-20 rpm |
| Spray solution rate: | 8-12 g/min |
| Inlet air flow: | 120 Nmⁿ/h |
| Inlet air temperature: | 40-45° C. |
| Tablet temperature: | 30-35° C. |

After 4 hours of process, the total weight gained by the active tablets was 28%.

Dissolution Profiles

The dissolution test was carried out by USP method (basket apparatus) using 900 ml Simulated Intestinal Fluid (SIF, pH 6.8) at 37° C., speed rotation was set at 100 rpm (Vankel system). Sampling was carried out at predetermined intervals and the drug was assayed by UV method (Cary spectrometer) and is expressed as percentage of the declared amount.

Results in vitro dissolution test (%)
(SIF pH 6.8, Baskets 100 rpm)

| | Time (hours) | | | | | | |
|---|---|---|---|---|---|---|---|
| % coating | 0 | 1 | 2 | 4 | 8 | 12 | 24 |
| 0% (uncoated tablets) | 0 | 30.1 | 43.3 | 59.2 | 77.1 | 86.4 | 95.2 |
| 28% | 0 | 8.8 | 22.8 | 40.1 | 64.9 | 79.5 | — |

Observations:

The uncoated tablets were intact at the end of the dissolution test. Coated tablets showed a coating layer around them after 24 hours of dissolution testing. This coating layer had swelled and these coated tablets were almost round with diameter dimensions of 8.7 to 9.4 mm. The core tablet was inside the swelled coating and maintained completely its integrity. These coated tablets exhibit a zero or near zero order release.

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

1. A pharmaceutical tablet composition comprising (i) a core, which comprises a lipophilic matrix and an effective amount of venlafaxine hydrochloride; and (ii) a water insoluble, permeable coating over said core.

2. The composition according to claim 1, wherein said lipophilic matrix is a fatty acid wax.

3. The composition according to claim 2, wherein said fatty acid wax is selected from the group consisting of palmitic acid, behenic acid, stearic acid, glyceryl behenate, glyceryl palmitostearate, hydrogenated castor oil, and combinations of two or more.

4. The composition according to claim 1, wherein said core further comprises an inert filler such as calcium phosphate and a lubricant.

5. The composition according to claim 1, wherein said coating comprises an acrylic polymer.

6. The composition according to claim 5, wherein said acrylic polymer is an acrylate-methacrylate copolymer.

7. The composition according to claim 5, wherein said acrylic polymer is an Eudragit RL.

8. The composition according to claim 1, which further comprises an enteric coat.

9. A process for making a modified release tablet, which comprises:
(i) granulating venlafaxine hydrochloride and a fatty acid wax to form granules;
(ii) compressing said granules optionally with additional excipients to form a tablet; and
(iii) forming a water insoluble, permeable coating around said tablet.

10. The process according to claim 9, wherein said fatty acid wax is selected from the group consisting of palmitic acid, behenic acid, stearic acid, glyceryl behenate, glyceryl palmitostearate, hydrogenated castor oil, and combinations of two or more.

11. The process according to claim 10, wherein said fatty acid wax is glyceryl behenate.

12. The process according to claim 9, wherein said coating comprises an acrylic polymer.

13. The process according to claim 12, wherein said forming step comprises coating said tablet with an acrylic latex and drying.

14. The process according to claim 9, which further comprises (iv) forming an enteric coating around said water insoluble, permeable coating.

15. A tablet produced according to the process of claim 9.

16. A process for making tablets, which comprises:
(a) co-processing a fatty acid wax with calcium phosphate to form a modified excipient;
(b) blending said modified excipient, a venlafaxine hydrochloride, and optionally one or more additional excipients to form a compression blend;
(c) compressing said compression blend to form a tablet; and
(d) coating said tablet with a water-insoluble, permeable coating.

17. The process according to claim 16, wherein said coating step comprises coating said tablet with an acrylic latex and drying.
